(19) United States
(12) Patent Application Publication
(10) Pub. No.: US 2019/0284171 A1
(43) Pub. Date: Sep. 19, 2019

(54) METHOD OF CONVERTING CARBON DIOXIDE INTO CARBONYL COMPOUNDS

(71) Applicant: TRANSLATIONAL HEALTH SCIENCE AND TECHNOLOGY INSTITUTE, Faridabad (IN)

(72) Inventors: Dinesh MAHAJAN, Faridabad (IN); Varun KUMAR, Faridabad (IN); Anil RANA, Faridabad (IN)

(21) Appl. No.: 16/346,625

(22) PCT Filed: Oct. 11, 2018
(86) PCT No.: PCT/IN2018/050648

§ 371 Date: May 1, 2019

(30) Foreign Application Priority Data
Oct. 12, 2017 (IN) 201711036319

(51) Int. Cl. C07D 403/06; C07C 237/52; C07D 417/06; C07D 413/06; C07D 401/06; C07D 295/14; C07D 295/04; C07D 221/00; C07D 285/15; C07D 317/38; C07C 49/175

(52) U.S. Cl. CPC C07D 403/06; C07C 237/52; C07D 417/06; C07D 413/06; C07D 401/06; C07C 49/175; C07D 295/04; C07D 221/00; C07D 285/15; C07D 317/38; C07D 295/14

(57) ABSTRACT

The present invention provides a method for fixing carbon dioxide gas as a carbonyl compound represented by formula (3) as depicted by FIG. 1 and comprising, purging of carbon dioxide in a solution of a nucleophile represented by the formula (1) in presence of a solvent at a temperature ranging from −40 Degree Celsius to 35 Degree Celsius, followed by adding a reagent at temperature ranging from −40 degree to 35 degree and thereafter adding another nucleophile represented by the formula (2) to obtain carbonyl compound represented by formula (3). The present invention can be advantageously used to obtain commercially important carbonyl compounds and clean unwanted carbon dioxide gas from the atmosphere and industrial effluents.

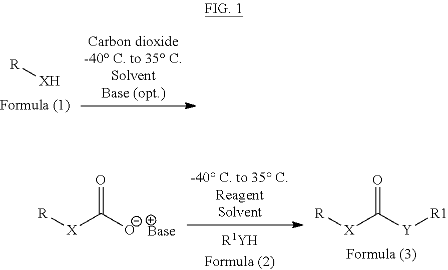

### FIELD OF THE INVENTION

The present invention relates to generally to the field of synthetic organic chemistry. In particular, the present application relates to chemical processes for use of carbon dioxide gas as a source of carbon to make carbonyl compounds.

### BACKGROUND OF THE INVENTION

Carbon dioxide (CO₂) is nontoxic, non-flammable, gas and colorless, odorless, incombustible gas, present in the atmosphere and formed during respiration, usually released during combustion of coal, coke, or natural or from cement processing plant either as effluent or as by product of the process. Carbon dioxide is also enormously produced synthetically from carbohydrates by fermentation, by reaction of acid with limestone or other carbonates. It is also produced naturally from springs.

Pure carbon dioxide is used extensively in industry as dry ice and carbon dioxide snow. Carbon dioxide use in carbonated beverage industry and fire extinguishers industry is almost inevitable. People and animals release carbon dioxide when they breathe out. Also, combustion of carbonyl compound produces carbon dioxide. The extra carbon dioxide in environment is detrimental and increases the greenhouse effect.

Increasingly dire warnings of the dangers of climate change by the world's scientific community combined with greater public awareness and concern over the issue has prompted increased momentum towards global regulation aimed at reducing man-made greenhouse gas (GHGs) emissions, most notably carbon dioxide. According to the International Energy Agency's (IEA) GHG Program, as of 2006 there were nearly 5,000 fossil fuel power plants worldwide generating nearly 11 billion tons of CO2, representing nearly 40% of total global anthropogenic CO2 emissions. Of these emissions from the power generation sector, 61% were from coal fired plants. Although the long-term agenda advocated by governments is replacement of fossil fuel generation by renewables, growing energy demand, combined with the enormous dependence on fossil generation in the near to medium term dictates that this fossil base remain operational. Thus, to implement an effective GHG reduction system will require that the CO2 emissions generated by this sector be mitigated, with carbon capture and storage (CCS) providing one of the best known solutions.

The CCS process removes CO2 from a CO2 containing flue gas, enables production of a highly concentrated CO2 gas stream which is compressed and transported to a sequestration site. This site may be a depleted oil field or a saline aquifer. Sequestration in ocean and mineral carbonation are two alternate ways to sequester that are in the research phase. Captured CO2 can also be used for enhanced oil recovery. But there are increasing evidences and arguments against this especially because of the poor energy efficiency and economy of the process involving transportation of captured CO2.

Chemical Fixation of carbon dioxide has attracted much attention in view of environmental, legal, and social issues in the past few decades. Carbon fixation or carbon assimilation is the conversion process of inorganic carbon (carbon dioxide) to carbonyl compounds. It is envisaged that a general method to utilise the carbon dioxide is as a reagent or reactant in synthetic chemistry can provide a better approach to tackle this greenhouse gas.

Carbon dioxide has been utilised as a solvent in supercritical fluid extraction, to convert CO2 and olefins into cyclic carbonates in water (Green Chem.2007, 9, 213), are useful and often greener substitutes for toxic phosgene (COCl₂) and dimethyl sulfate in a host of chemical reactions. These carbonates serve well as solvents, especially in medicines and cosmetics, and they are electrolytes of choice in lithium-ion batteries.

Carbon dioxide is a building block in organic synthesis because it is an abundant, renewable carbon source and an environmentally friendly chemical reagent. The utilization of carbon dioxide to useful bulk products is an economical one. Certain chemical reactions are available in prior art whereby the carbon-di-oxide is utilized to arrive at carbonyl compounds. However, the conditions are harsh and severe reaction and therefore their practical application is limited. A number of factors still need to be met, including reducing organic solvent use, reducing the number of reactants and reaction steps, reducing energy consumption, and reducing waste.

Further, as environmental regulations and safety concerns are the burgeoning issues faced by the industrial society today, development of environmentally benign methodologies remains the key issue. Hence, there is need to develop a simple, efficient, environmental friendly process for preparation of carbonyl compounds under mild conditions having varied industrial applications.

### SUMMARY OF THE INVENTION

In one aspect of the present invention, there is provided a method for fixing carbon dioxide gas as a carbonyl compound represented by formula (3) as depicted by FIG. 1 and comprising, purging of carbon dioxide in a solution of a nucleophile represented by the formula (1) in presence of a solvent at a temperature ranging from −40 Degree Celsius to 35 Degree Celsius, followed by adding a reagent at temperature ranging from −40 degree to 35 degree and thereafter adding another nucleophile represented by the formula (2) to obtain carbonyl compound represented by formula (3).

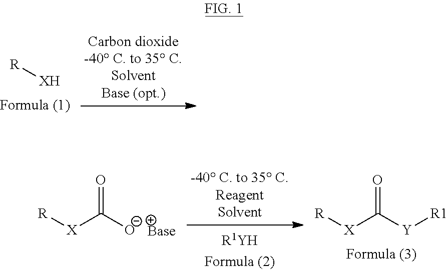

Wherein, X or Y is independently selected from the group comprising NR², O or S; Wherein, R, R¹ or R² is independently selected from the group comprising H, C₁-C₁₂ alkyl, C₃ to C₇ cyclic alkyl, C₄ to C₁₀ aryl, C₄ to C₁₀ heteroaryl comprising one or more heteroatoms selected from N, O, or S.

Wherein, R, R¹ or R² may be optionally substituted with one or more from the group comprising F, CI, Br, I, OR, NO₂, CN, N(R)₂, COOR, CON(R)₂, N(R)CON(R)₂, N(R)COR, N(R)SO₂N(R)₂, SO₂N(R)₂, SO₂R, SOR, SR, N(R)SO2R, aryl, heteroaryl, arylalkyl, (CH₂)ₘCO₂R (CH₂)ₘCO₂N(R)₂, C₂-C₁₂ alkenyloxy, C₂-C₁₂ alkynyloxy, C₂-C₁₂ heteroalkyloxy, C₃-C₈ cycloalkyloxy, C₃-C₈ cycloalkenyloxy, C₃-C₈ heterocycloalkyloxy, C₃-C₈ heterocycloalkenyloxy, and C₁-C₈ alkylamino.

Wherein, R and R² may form a C₃-C₆ cyclic ring, which may be further be optionally substituted with one or more of substituents selected from the group comprising F, CI, Br, I, OR, NO₂, CN, N(R)₂, COOR, CON(R)₂, N(R)CON(R)₂, N(R)COR, N(R)SO₂N(R)₂, SO₂N(R)₂, SO₂R, SOR, SR, N(R)SO₂R, aryl, heteroaryl, arylalkyl, (CH₂)ₘCO₂R (CH₂)ₘCO₂N(R)₂, C₂-C₁₂ alkenyloxy, C₂-C₁₂ alkynyloxy, C₂-C₁₂ heteroalkyloxy, C₃-C₈ cycloalkyloxy, C₃-C₈ cycloalkenyloxy, C₃-C₈ heterocycloalkyloxy, C₃-C₈ heterocycloalkenyloxy optionally substituted with C₁-C₈ alkylamino.

In another aspect of the present invention, there is provided a method for fixing carbon dioxide gas as a carbonyl compound represented by formula (3) as depicted in FIG. 2 below and comprising purging of carbon dioxide gas in a stirring solution containing nucleophile represented by formula (1) and nucleophile represented by formula (2) together in the solvent at a temperature ranging from −40° C. to 35° C., followed by slow addition of the reagent at a temperature ranging from −40° C. to 35° C. into the reaction mixture to give carbonyl compound represented by formula (3).

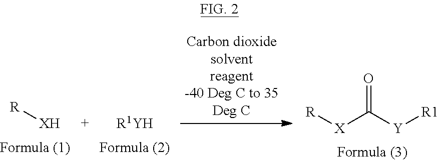

Wherein, X or Y is independently selected from the group comprising NR², O or S;

Wherein, R, R¹ or R² is independently selected from the group comprising H, C₁-C₁₂ alkyl, C₃ to C₇ cyclic alkyl, C₄ to C₁₀ aryl, C₄ to C₁₀ heteroaryl comprising one or more heteroatoms selected from N, O, or S.

Wherein, R, R¹ or R² may be optionally substituted with one or more from the group comprising F, CI, Br, I, OR, NO₂, CN, N(R)₂, COOR, CON(R)₂, N(R)CON(R)₂, N(R)COR, N(R)SO₂N(R)₂, SO₂N(R)₂, SO₂R, SOR, SR, N(R)SO₂R, aryl, heteroaryl, arylalkyl, (CH₂)ₘCO₂R (CH₂)ₘCO₂N(R)₂, C₂-C₁₂ alkenyloxy, C₂-C₁₂ alkynyloxy, C₂-C₁₂ heteroalkyloxy, C₃-C₈ cycloalkyloxy, C₃-C₈ cycloalkenyloxy, C₃-C₈ heterocycloalkyloxy, C₃-C₈ heterocycloalkenyloxy, and C₁-C₈ alkylamino.

Wherein, R and R² may form a C₃-C₆ cyclic ring, which may be further be optionally substituted with one or more of substituents selected from the group comprising F, CI, Br, I, OR, NO₂, CN, N(R)₂, COOR, CON(R)₂, N(R)CON(R)₂, N(R)COR, N(R)SO₂N(R)₂, SO₂N(R)₂, SO₂R, SOR, SR, N(R)SO₂R, aryl, heteroaryl, arylalkyl, (CH₂)ₘCO₂R (CH₂)ₘCO₂N(R)₂, C₂-C₁₂ alkenyloxy, C₂-C₁₂ alkynyloxy, C₂-C₁₂ heteroalkyloxy, C₃-C₈ cycloalkyloxy, C₃-C₈ cycloalkenyloxy, C₃-C₈ heterocycloalkyloxy, C₃-C₈ heterocycloalkenyloxy optionally substituted with C₁-C₈ alkylamino.

In yet another aspect of the present invention, there is provided a method for fixing carbon dioxide as carbonyl compound as depicted in FIG. 3 below and comprising purging of carbon dioxide in a premixed solution of the base and the reagent in the solvent for a period of 5 minutes to 30 minutes at a temperature ranging from −40° C. to 35° C., followed by addition of nucleophile represented by formula (1) and nucleophile represented by formula (2), either in two separate steps or simultaneously at a temperature ranging from −40° C. to 35° C. to obtain carbonyl compound represented by formula (3).

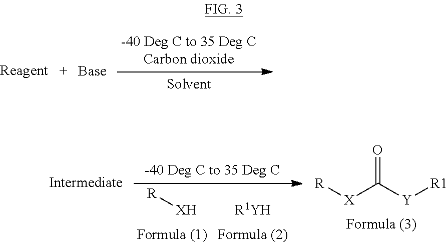

Wherein, X or Y is independently selected from the group comprising NR², O or S;

Wherein, R, R¹ or R² is independently selected from the group comprising H, C₁-C₁₂ alkyl, C₃ to C₇ cyclic alkyl, C₄ to C₁₀ aryl, C₄ to C₁₀ heteroaryl comprising one or more heteroatoms selected from N, O, or S.

Wherein, R, R¹ or R² may be optionally substituted with one or more from the group comprising F, CI, Br, I, OR, NO₂, CN, N(R)₂, COOR, CON(R)₂, N(R)CON(R)₂, N(R)COR, N(R)SO₂N(R)₂, SO₂N(R)₂, SO₂R, SOR, SR, N(R)SO₂R, aryl, heteroaryl, arylalkyl, (CH₂)ₘCO₂R (CH₂)ₘCO₂N(R)₂, C₂-C₁₂ alkenyloxy, C₂-C₁₂ alkynyloxy, C₂-C₁₂ heteroalkyloxy, C₃-C₈ cycloalkyloxy, C₃-C₈ cycloalkenyloxy, C₃-C₈ heterocycloalkyloxy, C₃-C₈ heterocycloalkenyloxy, and C₁-C₈ alkylamino.

Wherein, R and R² may form a C₃-C₆ cyclic ring, which may be further be optionally substituted with one or more of substituents selected from the group comprising F, CI, Br, I, OR, NO₂, CN, N(R)₂, COOR, CON(R)₂, N(R)CON(R)₂, N(R)COR, N(R)SO₂N(R)₂, SO₂N(R)₂, SO₂R, SOR, SR, N(R)SO₂R, aryl, heteroaryl, arylalkyl, (CH₂)ₘCO₂R (CH₂)ₘCO₂N(R)₂, C₂-C₁₂ alkenyloxy, C₂-C₁₂ alkynyloxy, C₂-C₁₂ heteroalkyloxy, C₃-C₈ cycloalkyloxy, C₃-C₈ cycloalkenyloxy, C₃-C₈ heterocycloalkyloxy, C₃-C₈ heterocycloalkenyloxy optionally substituted with C₁-C₈ alkylamino.

### DETAILED DESCRIPTION OF THE INVENTION

The present invention provides methods to fix carbon dioxide as carbonyl compounds. In one of the embodiment of the invention, carbon dioxide gas is purged in a stirring solution of a nucleophile represented by the formula (1) in a solvent at a temperature ranging from −40 Degree Celsius to 35 Degree Celsius, optionally with a base, followed by adding a reagent optionally with a base. Another nucleophile represented by the formula (2), optionally with a base is added to the above mixture to give the reaction product represented by formula (3).

In the above method purging of carbon dioxide or maintaining of CO2 atmosphere is optional after adding nucleophile represented by the formula (1).

Also, the nucleophile represented by the formula (1) and formula (2) shall be a nucleophile selected from a group consisting amine, alcohol or thiol.

The solvent which can be utilized in the above mentioned method can be any solvent suitable to be used with the nucleophile. Preferably, the solvent shall be one or more from the group of solvents consisting dichloromethane, dichloroetahne, THF, toluene, NMP, DMSO, water and dimethyl carbonate.

The nucleophile presented by formula (1) or (2) shall be optionally activated by one or more bases. The base used for this purpose is either organic or inorganic, depending upon the suitability with the nucleophile. Preferably the base is one or more selected from the group of bases consisting DBU, TEA, DIPEA, Pyridine, NaOH, NaH, sodium alkoxide, sodium carbonate, potassium carbonate, sodium bicarbonate and cesium carbonate.

The reagent which is utilized in the above mentioned method is selected from the group of reagents consisting POCl3, SOCl2, pTsCl, MsCl, Ms2O, oxalyl chloride, cyanuric chloride. The reagent is optionally with a base, either organic or inorganic, depending upon the suitability with the reagent. Preferably the base is one or more selected from the group of bases consisting DBU, TEA, DIPEA, Pyridine, NaOH, NaH, sodium alkoxide, sodium carbonate, potassium carbonate, sodium bicarbonate and cesium carbonate. The reaction under this embodiment is provided below as FIG. 1.

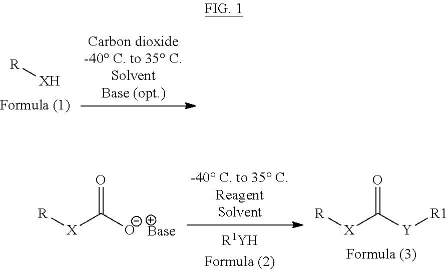

Wherein, X or Y is independently selected from the group comprising NR², O or S; Wherein, R, R¹ or R² is independently selected from the group comprising H, C₁-C₁₂ alkyl, C₃ to C₇ cyclic alkyl, C₄ to C₁₀ aryl, C₄ to C₁₀ heteroaryl comprising one or more heteroatoms selected from N, O, or S.

Wherein, R, R¹ or R² may be optionally substituted with one or more from the group comprising F, CI, Br, I, OR, NO₂, CN, N(R)₂, COOR, CON(R)₂, N(R)CON(R)₂, N(R)COR, N(R)SO₂N(R)₂, SO₂N(R)₂, SO₂R, SOR, SR, N(R)SO₂R, aryl, heteroaryl, arylalkyl, (CH₂)ₘCO₂R (CH₂)ₘCO₂N(R)₂, C₂-C₁₂ alkenyloxy, C₂-C₁₂ alkynyloxy, C₂-C₁₂ heteroalkyloxy, C₃-C₈ cycloalkyloxy, C₃-C₈ cycloalkenyloxy, C₃-C₈ heterocycloalkyloxy, C₃-C₈ heterocycloalkenyloxy, and C₁-C₈ alkylamino.

Wherein, R and R² may form a C₃-C₆ cyclic ring, which may be further be optionally substituted with one or more of substituents selected from the group comprising F, CI, Br, I, OR, NO₂, CN, N(R)₂, COOR, CON(R)₂, N(R)CON(R)₂, N(R)COR, N(R)SO₂N(R)₂, SO₂N(R)₂, SO₂R, SOR, SR, N(R)SO₂R, aryl, heteroaryl, arylalkyl, (CH₂)ₘCO₂R (CH₂)ₘCO₂N(R)₂, C₂-C₁₂ alkenyloxy, C₂-C₁₂ alkynyloxy, C₂-C₁₂ heteroalkyloxy, C₃-C₈ cycloalkyloxy, C₃-C₈ cycloalkenyloxy, C₃-C₈ heterocycloalkyloxy, C₃-C₈ heterocycloalkenyloxy optionally substituted with C₁-C₈ alkylamino.

In another embodiment of the present invention, carbon dioxide is purged in a stirring solution containing nucleophile represented by formula (1) and nucleophile represented by formula (2) together in the solvent at a temperature ranging from −40° C. to 35° C., optionally with the base. This is followed by slow addition of the reagent, optionally with the base, into the reaction mixture at a temperature ranging from −40° C. to 35° C.

In the above method purging of carbon dioxide or maintinaing a blanket of CO2 is optional after adding nucleophile.

Also, the nucleophile represented by the formula (1) and formula (2) shall be a nucleophile selected from a group consisting amine, alcohol or thiol.

The solvent which can be utilized in the above mentioned method can be any solvent suitable to be used with the nucleophile. Preferably, the solvent shall be one or more from the group of solvents consisting dichloromethane, dichloroetahne, THF, toluene, NMP, DMSO, water and dimethyl carbonate.

A base suitable to be used with the nucleophile presented by formula (1) or (2) is optionally present in this embodiment. The base used for this purpose is either organic or inorganic and preferably be one or more selected from the group of bases consisting DBU, TEA, DIPEA, Pyridine, NaOH, NaH, sodium alkoxide, sodium carbonate, potassium carbonate, sodium bicarbonate and cesium carbonate.

The reagent which is utilized in the above mentioned method is selected from the group of reagents consisting POCl3, SOCl2, pTsCl, MsCl, Ms2O, oxalyl chloride, cyanuric chloride. The reagent is optionally with a base, either organic or inorganic, depending upon the suitability with the reagent. Preferably the base is one or more selected from the group of bases consisting DBU, TEA, DIPEA, Pyridine, NaOH, NaH, sodium alkoxide, sodium carbonate, potassium carbonate, sodium bicarbonate and cesium carbonate.

The reaction under this embodiment is provided below as FIG. 2.

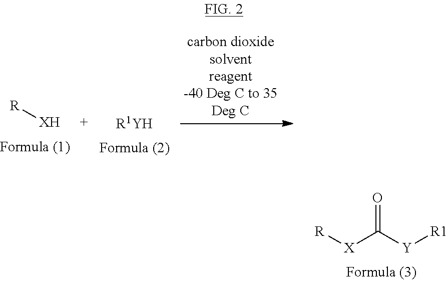

In yet another embodiment of the present invention, carbon dioxide is purged in a premixed solution of the base and the reagent in the solvent for a period of 5 minutes to 30 minutes at a temperature ranging from −40° C. to 35° C., followed by addition of nucleophile represented by formula (1) and nucleophile represented by formula (2) optionally in base, either in two separate steps or simultaneously at a temperature ranging from −40° C. to 35° C.

In the above method purging of carbon dioxide or maintaining a blanket of CO2 atmosphere is optional after adding nucleophile.

Also, the nucleophile represented by the formula (1) and formula (2) shall be a nucleophile selected from a group consisting amine, alcohol or thiol.

The solvent which can be utilized in the above mentioned method can be any solvent suitable to be used with the nucleophile. Preferably, the solvent shall be one or more from the group of solvents consisting dichloromethane, dichloroetahne, THF, toluene, NMP, DMSO, water and dimethyl carbonate.

A base suitable to be used with the nucleophile presented by formula (1) or (2) is optionally present in this embodiment. The base used for this purpose is either organic or inorganic and preferably be one or more selected from the group of bases consisting DBU, TEA, DIPEA, Pyridine, NaOH, NaH, sodium alkoxide, sodium carbonate, potassium carbonate, sodium bicarbonate and cesium carbonate.

The reagent which is utilized in the above mentioned method is selected from the group of reagents consisting POCl3, SOCl2, pTsCl, MsCl, Ms2O, oxalyl chloride, cyanuric chloride. The reagent is optionally with a base, either organic or inorganic, depending upon the suitability with the reagent. Preferably the base is one or more selected from the group of bases consisting DBU, TEA, DIPEA, Pyridine, NaOH, NaH, sodium alkoxide, sodium carbonate, potassium carbonate, sodium bicarbonate and cesium carbonate.

The reaction under this embodiment is provided below as FIG. 3.

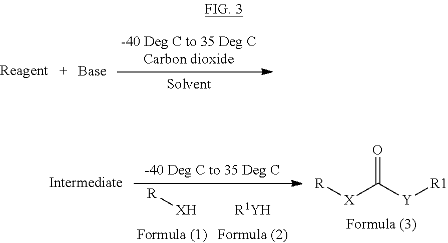

The present invention provides methods to fix carbon dioxide into various commercially important carbonyl compounds including but not limiting to isopoturon, carbaryl, carbosulfan, carbendazm, nithiazide, pyriminil, aminoquinuride, dimelitan, isolan, primacarb, triazamate, neostigmine, pyridostigmine, camazepam, dimethyl carbonate, diethyl carbonate, ethylene, carbonate, and dibenzyl carbonate etc. as provided below with indicated yield:

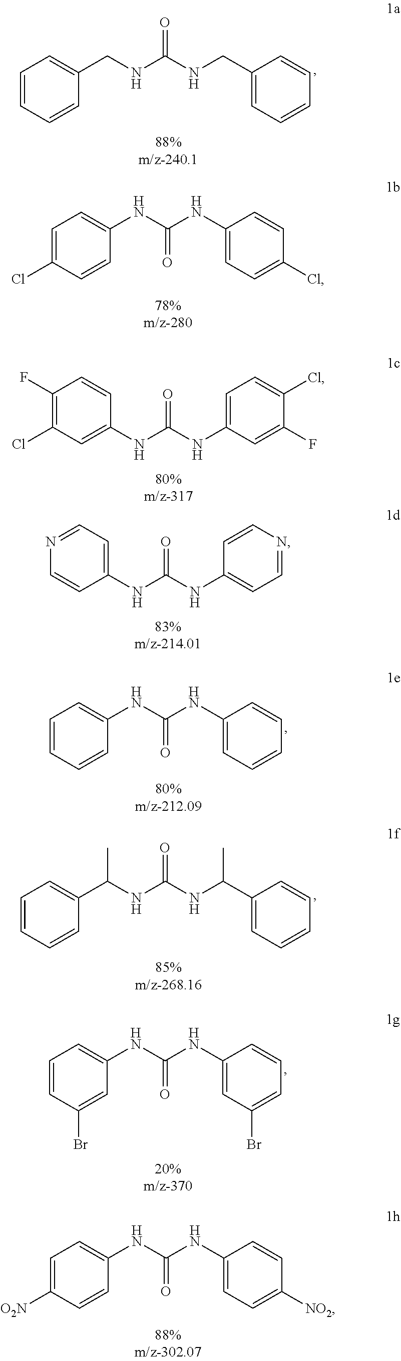

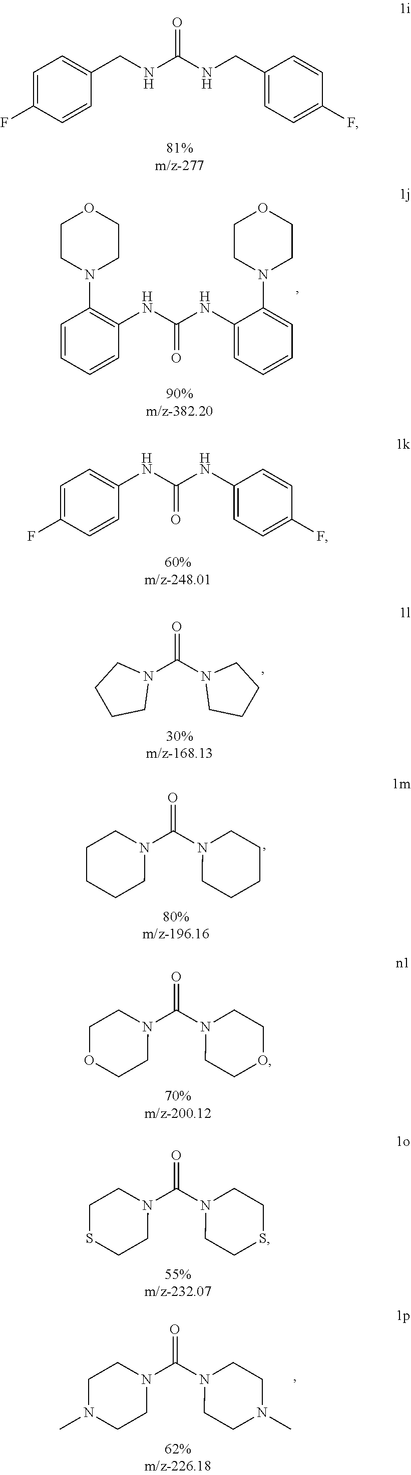

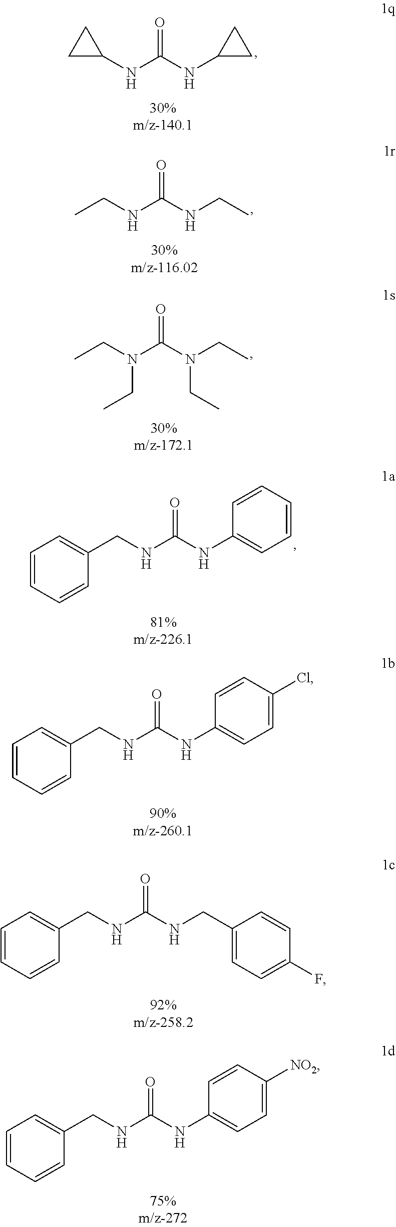

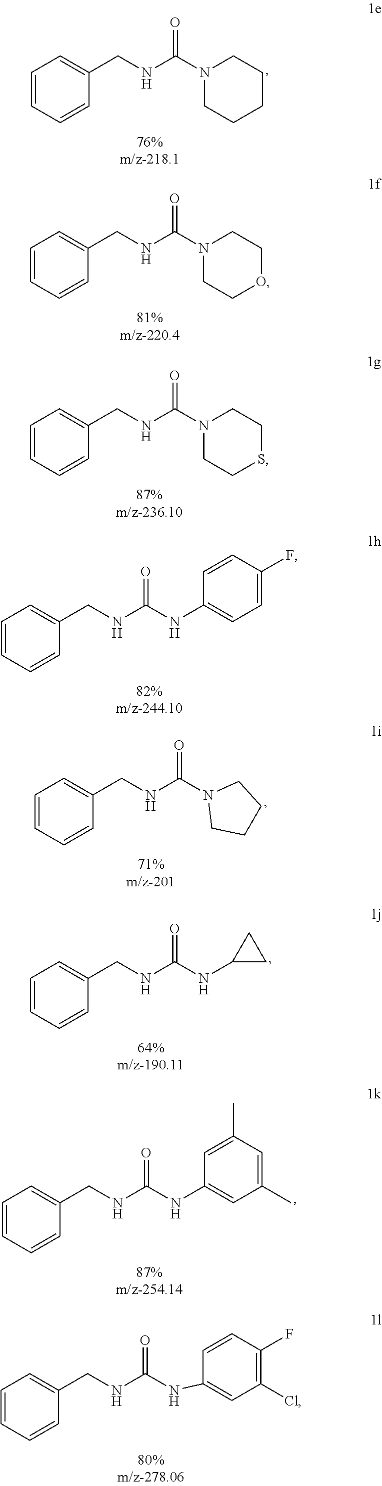

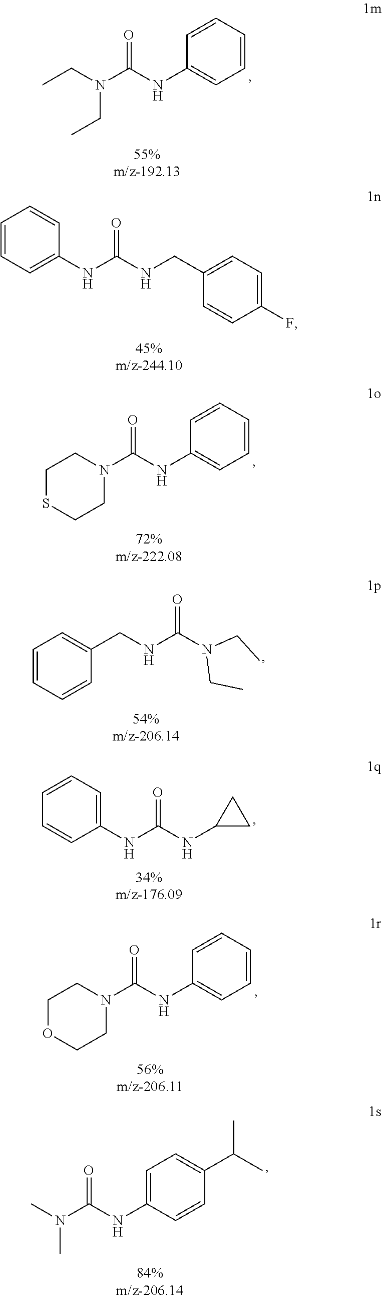

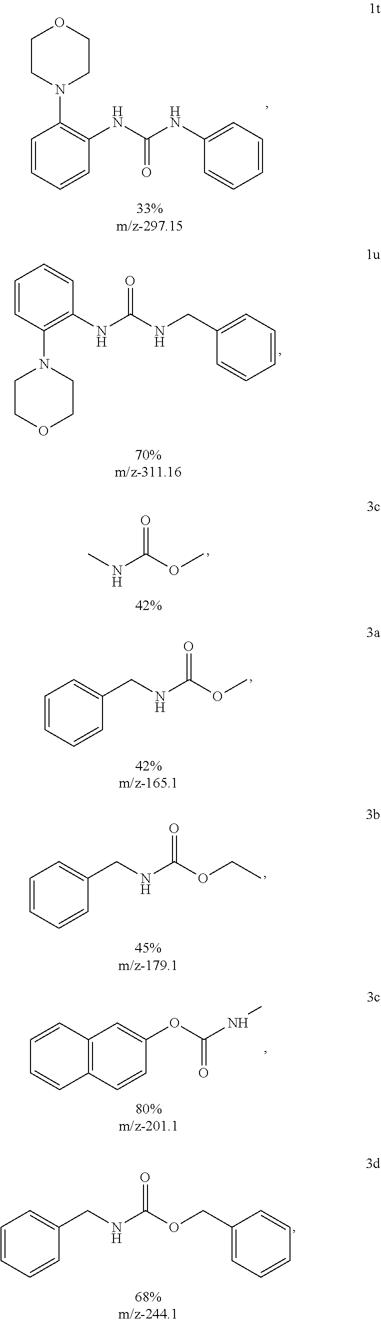

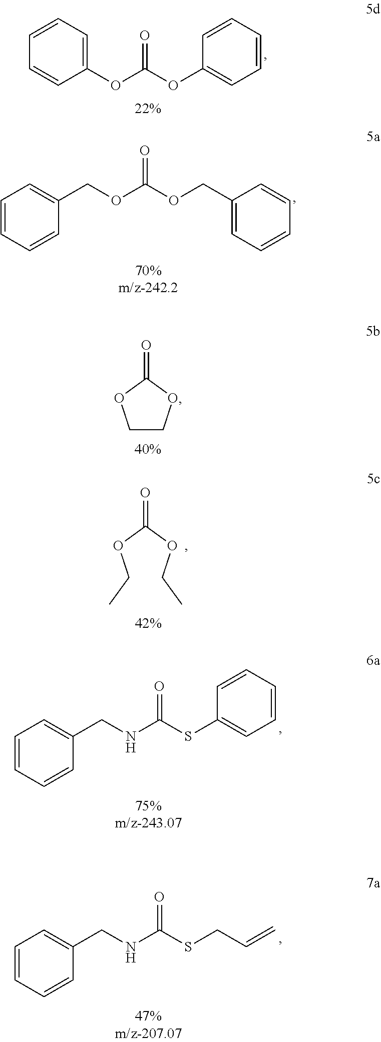

In one of the advantageous feature of the present invention, atmospheric carbon dioxide is fixed as commercially important carbonyl compound using method of the present invention. In another advantageous feature of the present invention, carbon dioxide gas in the industrial effluent is fixed as commercially important carbonyl compound using method of the present invention.

Hence, the present invention has two-fold advantage, firstly safeguarding our atmosphere from unwanted carbon dioxide gas and secondly providing commercially important carbonyl compound.

The process of the present invention may be illustrated by examples as set out herein below which should not be construed as limiting. The examples are only for illustrative purposes and are to be construed as exemplifications of the principle set out in the present application.

General Procedures:

The process of the present invention may be conducted by either of the process as below or by small changes or new adaptations:

General Procedure 1a:

CO2 may be purged in a stirring solution of a desired nucleophile or RXH that can be amine, alcohol or thiol; in a suitable solvent such as dichloromethane (DCM), Dichloroetahne (DCE), THF, Toluene, NMP, DMSO, water or dimethyl carbonate or combination of these but not limited to these; at a temperature ranging from −40 to 35 degree with or without presence of an external organic or inorganic base such as DBU, TEA, DIPEA, Pyridine, NaOH, NaH, sodium alkoxide, sodium carbonate, potassium carbonate, sodium bicarbonate and cesium carbonate but not limited to these. CO2 purging or a blanket of CO2 may or may not be sustained as required and a slow addition of a suitable reagent such as POCl3, SOCl2, pTsCl, MsCl, Ms2O, oxalyl chloride, cyanuric chloride as set out herein may be conducted in presence or absence of a suitable organic or inorganic base as mentioned above at a temperature ranging from −40 to 35 degree. This may be followed by addition second desired nucleophile R1YH that can be an amine, or alcohol or a thiol with and without activation with a suitable inorganic or organic base, and with or without sustaining the purging of CO2 or maintaining a blanket of CO2 atmosphere. The reaction mixture may be maintained at same temperature and may be heated if required so. Reaction progress may be monitored by suitable methods followed by isolation of the desired product.

General Procedure 1b:

In this case, CO2 may be purged in a stirring solution of a mixture of two or more desired nucleophiles ie. RXH and R1YH that can be amine, alcohol or thiol; in a suitable solvent such as dichloromethane (DCM), Dichloroetahne (DCE), THF, Toluene, NMP, DMSO, water, dimethyl carbonate or combination of these but not limited to these; at a temperature ranging from −40 to 35 degree with or without presence of an external organic or inorganic base such as DBU, TEA, DIPEA, Pyridine, NaOH, NaH, sodium alkoxide, sodium carbonate, potassium carbonate, sodium bicarbonate and cesium carbonate but not limited to these. CO2 purging or a blanket of CO2 may or may not be sustained as required and a slow addition of a suitable reagent such as POCl3, SOCl2, pTsCl, MsCl, Ms2O, oxalyl chloride, cyanuric chloride as set out herein may be. The reaction mixture may be maintained at same temperature and may be heated if required so. Reaction progress may be monitored by suitable methods followed by isolation of the desired product.

General Procedure 1c

In other case, the suitable base such as DBU, TEA, DIPEA, Pyridine, NaOH, NaH, sodium alkoxide, sodium carbonate, potassium carbonate, sodium bicarbonate and cesium carbonate but not limited to these; and suitable reagent such as POCl3, SOCl2, pTsCl, MsCl, Ms2O, oxalyl chloride, cyanuric chloride; can be premixed in a suitable solvent such as dichloromethane (DCM), Dichloroetahne (DCE), THF, Toluene, NMP, DMSO, water dimethyl carbonate or combination of these but not limited to these. CO2 purging may be done for a period of 5-30 minutes followed by addition of two nucleophiles (RXH and R1YH) step wise or together with or without any additional organic or inorganic base at a temperature ranging from −40 to 35 degree celsius.

General Procedure for Synthesis of Symmetric Ureas:

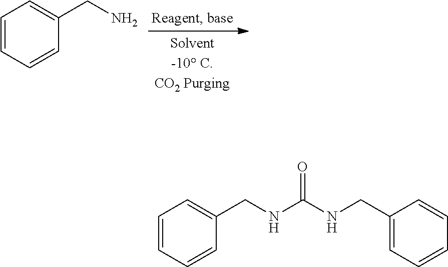 [SMILES: NCc1ccccc1 ; O=C(NCc1ccccc1)NCc1ccccc1]

To a premixed solution of a suitable base such as DBU or DIPEA or sodium carbonate preferably DIPEA; and appropriate amine such as benzyl amine in a suitable solvent such DCE or DCM; was purged CO2 gas for some time ranging from 5 minutes to 30 minutes. An addition of a reagent such as POCl3 or MsCl or TsCl was done at a temperature range between −40 degree to 35 degree but preferably at −20 degree Celsius for activation to afford the corresponding urea and progress of reaction was monitored by TLC. After completion of reaction product was obtained by aqueous work up with organic solvent without any further purification such as column chromatography or crystalization.

General Procedure for Synthesis of Mixed Ureas:

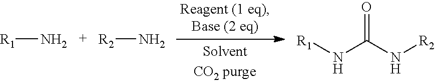

In this case, CO2 was purged in a stirring solution of a mixture of two different amines; in a suitable solvent such as dichloromethane; in presence of an external base such as DIPEA. This was followed by a slow addition of a suitable reagent such as POCl3 at a temperature ranging from −40 to 35 degree. The reaction mixture may be maintained at same temperature for some time before bringing to room temperature and reaction progress may be monitored by suitable method known in art such as TLC or HPLC. The reaction was worked up by method known in art followed by purification using solvent washing, crystallization of column chromatography to get desired product.

General Procedure for Synthesis of Carbamates and Thiocarbamates:

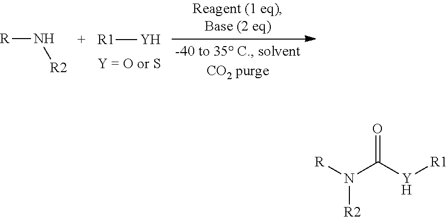

In this case, CO2 was purged in a stirring solution of a mixture of amine and alcohol; in a suitable solvent such as dichloromethane (DCM); with a base such as DIPEA. CO2 purging may or may not be sustained as required and a slow addition of a suitable reagent such as POCl3 at a temperature ranging from −40 to 35 degree. The reaction mixture was allowed to stay at same temperature for some time before bringing to RT. Reaction progress was monitored by suitable methods followed by isolation of the desired product.

The above reported procedure may also be use for synthesis of thio-carbamates and dicarbonates.

### EXAMPLES

### Example 1

### Synthesis of Dibenzyl Urea

In one method, carbon dioxide is purged in a solution of diisopropylethylamine (2 eq., 2 mmol) and p-toluene sulphonyl chloride (1 eq., 1 mmol) in dichloromethane for 30 minutes at room temperature. To this 1 eq. benzylamine is added dropwise at 0° C. with continuous purging of carbon dioxide. On completion of the reaction, reaction mixture is diluted with water and extracted in to 60 mL of ethyl acetate (60 mL), followed by first wash with 1N HCL (5 mL) and second wash with a mixture of NaHCO₃ (10 mL) and brine (10 mL). Combined organic phases were dried over Na₂SO₄ and concentrated under reduced pressure to give dibenzyl urea which is further purified by column chromatography to obtain dibenzyl urea with 88% yield (366 mg)

The above procedure can also be performed by replacing POCl3 by other reagents and different bases as mentioned in following table 1.

TABLE 1

| Entry | Reagent | Base | Solvent | Yield (%) |
|---|---|---|---|---|
| 1 | POCl3 | Et3N | DCM | 70% |
| 2 | MsCl | Et3N | DCM | 50% |
| 3 | Ms2O | Et3N | DCM | 59% |
| 4 | p-TsCl | Et3N | DCM | 83% |
| 5 | p-TsCl | DIPEA | DCM | 88% |
| 6 | p-TsCl | Pyridine | DCM | 56% |

In another method, 2.2 eq. (4.4 mmol) of di-isopropylethylamine is added into a solution of benzyl amine (2 eq, 4 mmol) in DCM (10 mL) and carbon dioxide is purged through the solution for 30 minutes at 0° C., followed by addition of 1.1 eq (2.2 mmol) of POCl3. On completion, reaction mixture is diluted with dichloromethane and washed first with 1N HCl and secondly with brine. Combined organic phases were dried over Na₂SO4 and concentrated to give the dibenzyl urea which is further purified by column chromatography if necessary.

Methods disclosed in Example 1 are used to synthesize under mentioned carbonyl compounds:

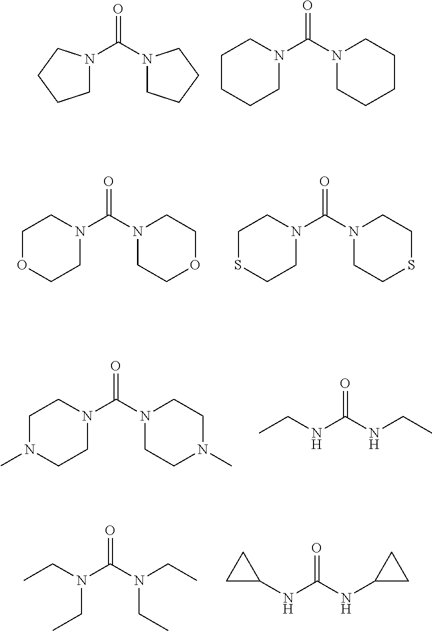

### Example 2

### Synthesis of 1-Benzyl-3-phenylurea

In one method, 2.2 eq, 4.4 mmol of di-isopropylethylamine is added to a mixture of benzyl amine (1 eq, 2 mmol) and aniline (2 eq, 4 mmol) in dichloromethane (10 mL) and carbon dioxide is purged through the reaction mixture for 30 minutes, followed by addition of 1.1 eq, 2.2 mmol of POCl3. On completion, the reaction mixture is diluted with dichloromethane and washed firstly with 1N HCl and secondly with brine. Combined organic phase is dried over Na2SO4 and concentrated to give 1-Benzyl-3-phenylurea, which was further purified by column chromatography to obtain 366 mg of 1-Benzyl-3-phenylurea (81% yield).

Methods disclosed in Example 2 are used to synthesize under mentioned carbonyl compounds with indicated percentage yields:

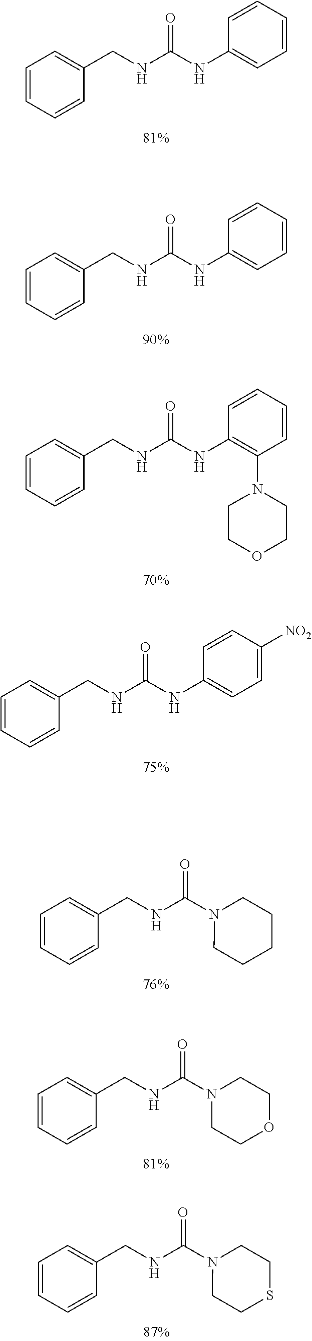

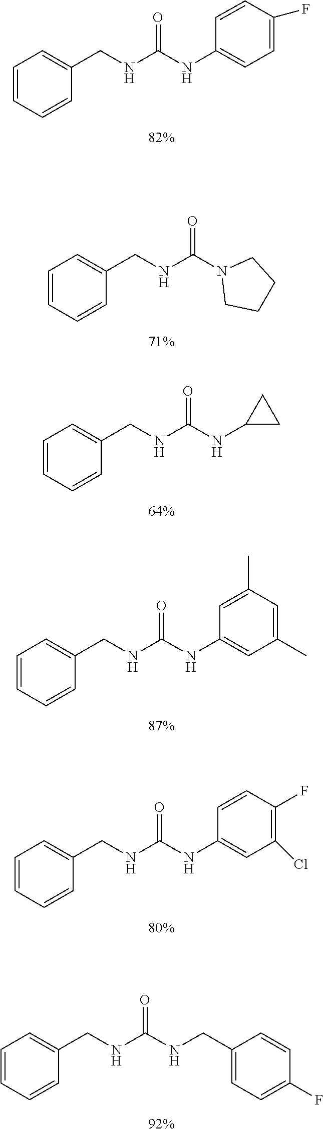 [SMILES: O=C(NCc1ccccc1)Nc1ccc(F)cc1 ; O=C(NCc1ccccc1)N1CCCC1 ; O=C(NCc1ccccc1)NC1CC1 ; Cc1cc(C)cc(NC(=O)NCc2ccccc2)c1 ; O=C(NCc1ccccc1)Nc1ccc(F)c(Cl)c1 ; O=C(NCc1ccccc1)NCc1ccc(F)cc1]

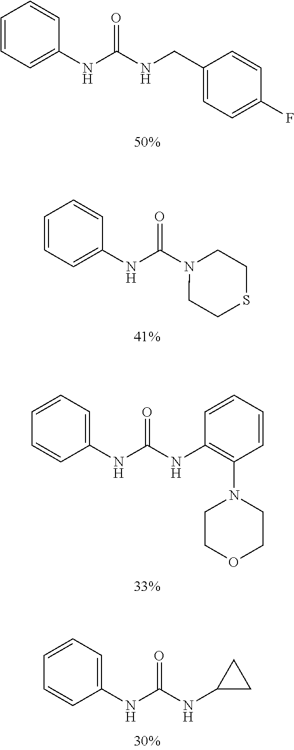 [SMILES: O=C(NCc1ccc(F)cc1)Nc1ccccc1 ; O=C(Nc1ccccc1)N1CCSCC1 ; O=C(Nc1ccccc1)Nc1ccccc1N1CCOCC1 ; O=C(Nc1ccccc1)NC1CC1]

### Example 3

### Synthesis of Benzyl benzylcarbamate

In one method, 2.2 eq, 4.4 mmol of di-isopropylethylamine is added to the mixture of benzyl amine (1 eq, 2 mmol) and benzyl alcohol (2 eq, 4 mmol) in dichloromethane (10 mL), followed by purging with carbon dioxide for 30 minutes and addition of 1.1 eq, 2.2 mmol of POCl3 thereafter. On completion, reaction mixture is diluted with dicholormethane and washed firstly with water and secondly with brine. Combined organic phase is dried over Na2SO4, concentrated and purified by column chromatography to obtain ? mg of Benzyl benzylcarbamate with 68% yields (330 mg).

### Example 4

### Synthesis of S-Phenyl benzylcarbamothioate

2.2 eq, 4.4 mmol of di-isopropylethylamine is added to the mixture of benzylamine (1 eq, 2 mmol) and thioalcohol (2 eq, 4 mmol) in dichloromethane (10 mL) and carbon dioxide is purged through the solution for 30 minutes, followed by addition of 1.1 eq, 2.2 mmol POCl3. On completion, the reaction mixture is diluted with dichloromethane and washed firstly with water and secondly with brine. Combined organic phase is dried over Na2SO4 and concentrated to give S-Phenyl benzylcarbamothioate which is further purified by solvent washing to obtain ? mg of S-Phenyl benzylcarbamothioate with 68% yields (332 mg). This method is also utilized to prepare under mentioned compound with 194 mg..

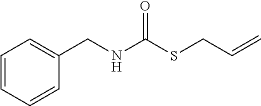

### Example 5

### Synthesis of dibenzyl carbonate

2.2 eq, 4.4 mmol of di-isopropylethylamine is added to a solution of benzyl alcohol (2 eq, 4 mmol) in dicholomethane (10 mL) and carbon dioxide is purged through the solution for 30 minutes followed by addition of 1.1 eq, 2.2 mmol POCl3. On completion, the reaction mixture was diluted with dichloromethane and washed first with water and secondly with brine. Combined organic phase is dried over Na2SO4 and concentrated to give dibenzyl carbonate, which is further purified by column chromatography to obtain 338 mg of dibenzyl carbonate.

This method is also utilized to prepare under mentioned compounds with indicated yields:

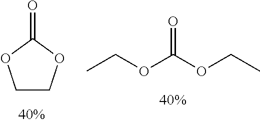

### Example 6

### Synthesis of 1-Naphthyl Methylcarbamate (Carbaryl)

2.2 eq, 2.2 mmol of di-isopropylethylamine is added to a solution of methylamine (4 eq, 4 mmol) in dichloromethane (20 mL) and carbon dioxide is purged through the solution for 45 minutes at −40° C. followed by addition of 1.1 eq, 1.1 mmol POCl3 and 1 eq, 1 mmol of α-naphthol. The reaction mixture is stirred for 2 minutes to 5 minutes, followed by addition of 1.1 eq. of 1,8-Diazabicyclo[5.4.0]undec-7-ene. On completion, the reaction mixture is diluted with dichloromethane and washed first with water, then by brine. The combined organic phase is dried over Na2SO4 and concentrated to give carbaryl, which was further purified by column chromatography to obtain 160 mg (80% yield) of carbaryl.

### Example 7

### Synthesis of N-(4-Isopropylphenyl)-N′,N′-dimethylurea (Isoproturon)

2.2 eq, 2.2 mmol of di-isopropylethylamine is added to the solution of dimethylamine (4 eq, 4 mmol) in dichloromethane (20 mL) and carbon dioxide is purged through the solution for 45 minutes at −40° C. followed by addition of 1.1 eq, 1.1 mmol POCl3 and 1 eq, 1 mmol 4-isopropylaniline. On completion, the reaction mixture is diluted with dichloromethane and washed first with water, then by brine. The combined organic phase is dried over Na2SO4 and concentrated to give Isoproturon, which was further purified by column chromatography to obtain 173 mg (84%) of Isoproturon with >90 purity.

We claim:

1. A method for fixing carbon dioxide gas as a carbonyl compound represented by formula (3) as depicted by reaction scheme provided in FIG. 1 below and comprising, purging of carbon dioxide in a stirring solution of a nucleophile represented by the formula (1) in presence of a solvent, followed by adding a reagent and thereafter adding another nucleophile represented by the formula (2) to obtain carbonyl compound represented by formula (3). 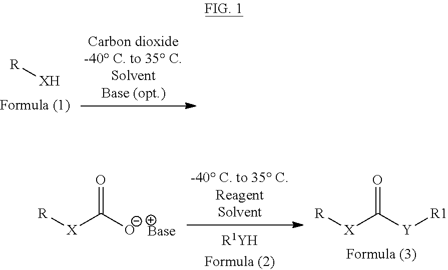
Wherein, X or Y is independently selected from the group comprising NR², O or S;
Wherein, R, R¹ or R² is independently selected from the group comprising H, C₁-C₁₂ alkyl, C₃ to C₇ cyclic alkyl, C₄ to C₁₀ aryl, C₄ to C₁₀ heteroaryl comprising one or more heteroatoms selected from N, O, or S.
Wherein, R, R¹ or R² may be optionally substituted with one or more from the group comprising F, CI, Br, I, OR, NO₂, CN, N(R)₂, COOR, CON(R)₂, N(R)CON(R)₂, N(R)COR, N(R)SO₂N(R)₂, SO₂N(R)₂, SO₂R, SOR, SR, N(R)SO₂R, aryl, heteroaryl, arylalkyl, (CH₂)ₘCO₂R (CH₂)ₘCO₂N(R)₂, C₂-C₁₂ alkenyloxy, C₂-C₁₂ alkynyloxy, C₂-C₁₂ heteroalkyloxy, C₃-C₈ cycloalkyloxy, C₃-C₈ cycloalkenyloxy, C₃-C₈ heterocycloalkyloxy, C₃-C₈ heterocycloalkenyloxy, and C₁-C₈ alkylamino.
Wherein, R and R² may form a C₃-C₆ cyclic ring, which may be further be optionally substituted with one or more of substituents selected from the group comprising F, CI, Br, I, OR, NO₂, CN, N(R)₂, COOR, CON(R)₂, N(R)CON(R)₂, N(R)COR, N(R)SO₂N(R)₂, SO₂N(R)₂, SO₂R, SOR, SR, N(R)SO₂R, aryl, heteroaryl, arylalkyl, (CH₂)ₘCO₂R (CH₂)ₘCO₂N(R)₂, C₂-C₁₂ alkenyloxy, C₂-C₁₂ alkynyloxy, C₂-C₁₂ heteroalkyloxy, C₃-C₈ cycloalkyloxy, C₃-C₈ cycloalkenyloxy, C₃-C₈ heterocycloalkyloxy, C₃-C₈ heterocycloalkenyloxy optionally substituted with C₁-C₈ alkylamino.

2. A method for fixing carbon dioxide gas as a carbonyl compound represented by formula (3) as depicted in reaction mechanism provided below as FIG. 2 and comprising purging of carbon dioxide gas in a stirring solution containing nucleophile represented by formula (1) and nucleophile represented by formula (2) together in a solvent, followed by slow addition of the reagent to give carbonyl compound represented by formula (3). 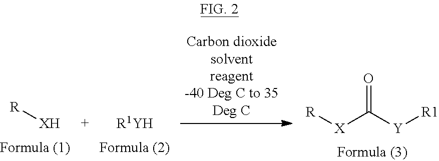
Wherein, X or Y is independently selected from the group comprising NR², O or S;
Wherein, R, R¹ or R² is independently selected from the group comprising H, C₁-C₁₂ alkyl, C₃ to C₇ cyclic alkyl, C₄ to C₁₀ aryl, C₄ to C₁₀ heteroaryl comprising one or more heteroatoms selected from N, O, or S.
Wherein, R, R¹ or R² may be optionally substituted with one or more from the group comprising F, CI, Br, I, OR, NO₂, CN, N(R)₂, COOR, CON(R)₂, N(R)CON(R)₂, N(R)COR, N(R)SO₂N(R)₂, SO₂N(R)₂, SO₂R, SOR, SR, N(R)SO₂R, aryl, heteroaryl, arylalkyl, (CH₂)ₘCO₂R (CH₂)ₘCO₂N(R)₂, C₂-C₁₂ alkenyloxy, C₂-C₁₂ alkynyloxy, C₂-C₁₂ heteroalkyloxy, C₃-C₈ cycloalkyloxy, C₃-C₈ cycloalkenyloxy, C₃-C₈ heterocycloalkyloxy, C₃-C₈ heterocycloalkenyloxy, and C₁-C₈ alkylamino.
Wherein, R and R² may form a C₃-C₆ cyclic ring, which may be further be optionally substituted with one or more of substituents selected from the group comprising F, CI, Br, I, OR, NO₂, CN, N(R)₂, COOR, CON(R)₂, N(R)CON(R)₂, N(R)COR, N(R)SO₂N(R)₂, SO₂N(R)₂, SO₂R, SOR, SR, N(R)SO₂R, aryl, heteroaryl, arylalkyl, (CH₂)ₘCO₂R (CH₂)ₘCO₂N(R)₂, C₂-C₁₂ alkenyloxy, C₂-C₁₂ alkynyloxy, C₂-C₁₂ heteroalkyloxy, C₃-C₈ cycloalkyloxy, C₃-C₈ cycloalkenyloxy, C₃-C₈ heterocycloalkyloxy, C₃-C₈ heterocycloalkenyloxy optionally substituted with C₁-C₈ alkylamino.

3. A method for fixing carbon dioxide as carbonyl compound as depicted in the reaction scheme provided below as FIG. 3 and comprising purging of carbon dioxide in a premixed solution of the base and the reagent in the solvent for a period of 5 minutes to 30 minutes, followed by addition of nucleophile represented by formula (1) and nucleophile represented by formula (2), either in two separate steps or simultaneously at a temperature ranging from to obtain carbonyl compound represented by formula (3). 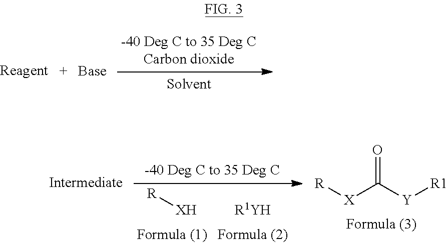
Wherein, X or Y is independently selected from the group comprising NR², O or S;
Wherein, R, R¹ or R² is independently selected from the group comprising H, C₁-C₁₂ alkyl, C₃ to C₇ cyclic alkyl, C₄ to C₁₀ aryl, C₄ to C₁₀ heteroaryl comprising one or more heteroatoms selected from N, O, or S.
Wherein, R, R¹ or R² may be optionally substituted with one or more from the group comprising F, CI, Br, I, OR, NO₂, CN, N(R)₂, COOR, CON(R)₂, N(R)CON(R)₂, N(R)COR, N(R)SO₂N(R)₂, SO₂N(R)₂, SO₂R, SOR, SR, N(R)SO2R, aryl, heteroaryl, arylalkyl, (CH₂)ₘCO₂R (CH₂)ₘCO₂N(R)₂, C₂-C₁₂ alkenyloxy, C₂-C₁₂ alkynyloxy, C₂-C₁₂ heteroalkyloxy, C₃-C₈ cycloalkyloxy, C₃-C₈ cycloalkenyloxy, C₃-C₈ heterocycloalkyloxy, C₃-C₈ heterocycloalkenyloxy, and C₁-C₈ alkylamino.
Wherein, R and R² may form a C₃-C₆ cyclic ring, which may be further be optionally substituted with one or more of substituents selected from the group comprising F, CI, Br, I, OR, NO₂, CN, N(R)₂, COOR, CON(R)₂, N(R)CON(R)₂, N(R)COR, N(R)SO₂N(R)₂, SO₂N(R)₂, SO₂R, SOR, SR, N(R)SO₂R, aryl, heteroaryl, arylalkyl, (CH₂)ₘCO₂R (CH₂)ₘCO₂N(R)₂, C₂-C₁₂ alkenyloxy, C₂-C₁₂ alkynyloxy, C₂-C₁₂ heteroalkyloxy, C₃-C₈ cycloalkyloxy, C₃-C₈ cycloalkenyloxy, C₃-C₈ heterocycloalkyloxy, C₃-C₈ heterocycloalkenyloxy optionally substituted with C₁-C₈ alkylamino.

4. A method of claims 1-3, wherein the method is performed at a temperature ranging from −40° C. to 35° C.

5. A method of claims 1-4, wherein the method is performed with continuous purging of carbon dioxide and with a blanket of CO2 atmosphere.

6. A method of claims 1-5, wherein solvent is one or more selected from chloromethane, dichloroetahne, THF, toluene, NMP, DMSO, water and dimethyl carbonate.

7. A method of claims 1-6, wherein the nucleophile or reagent is optionally activated by one or more bases selected from DBU, TEA, DIPEA, Pyridine, NaOH, NaH, sodium alkoxide, sodium carbonate, potassium carbonate, sodium bicarbonate and cesium carbonate.

8. A method of claims 1-7, wherein the reagent is one or more selected from POCl3, SOCl2, pTsCl, MsCl, Ms2O, oxalyl chloride, and cyanuric chloride.

9. A method of claims 1-7, wherein the reagent is POCl3 and base in DIPEA.

10. A method of claims 1-7, wherein reagent is POCl3, base is DIPEA and solvent is chloromethane.

11. A method of claim 1-10, wherein the carbon dioxide gas is used directly from industrial effluents rich in carbon dioxide, from atmospheric air, and commercially available purified CO2 gas cylinders.

12. A method of claim 1-11, wherein the method is used to synthesize one or more carbonyl compounds selected from: 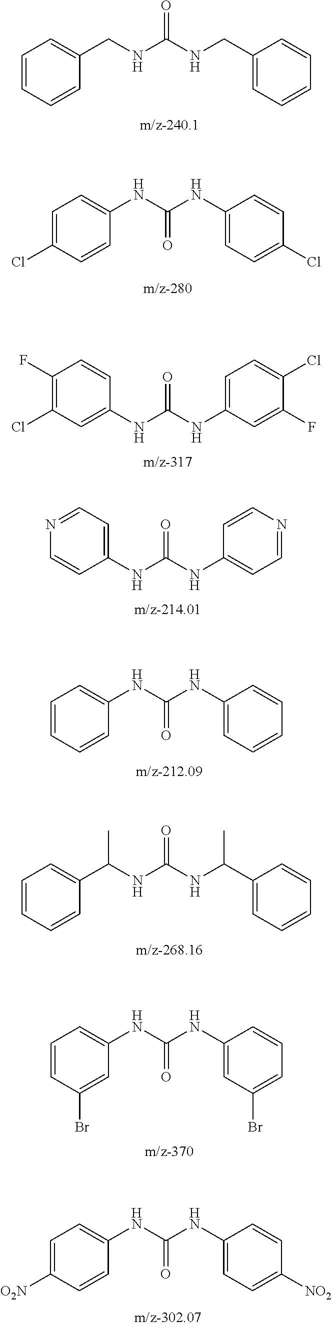 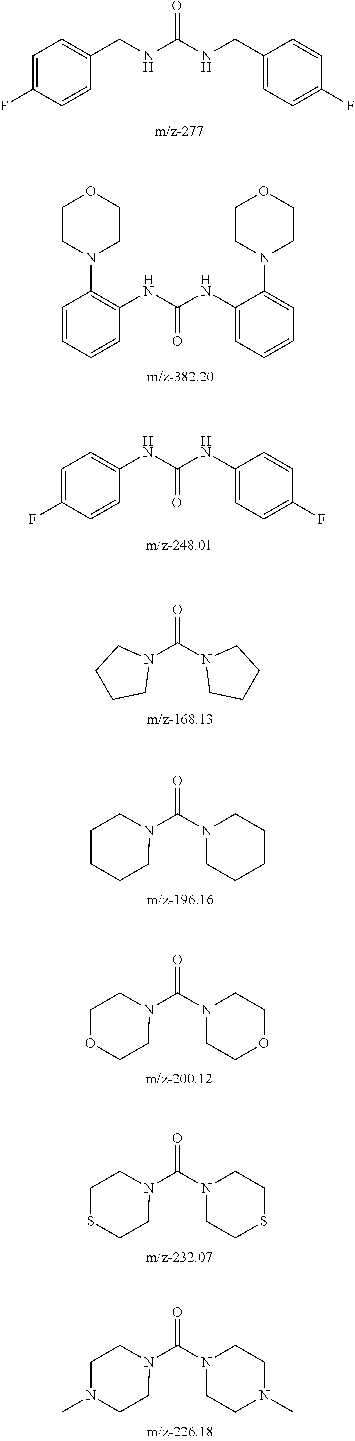 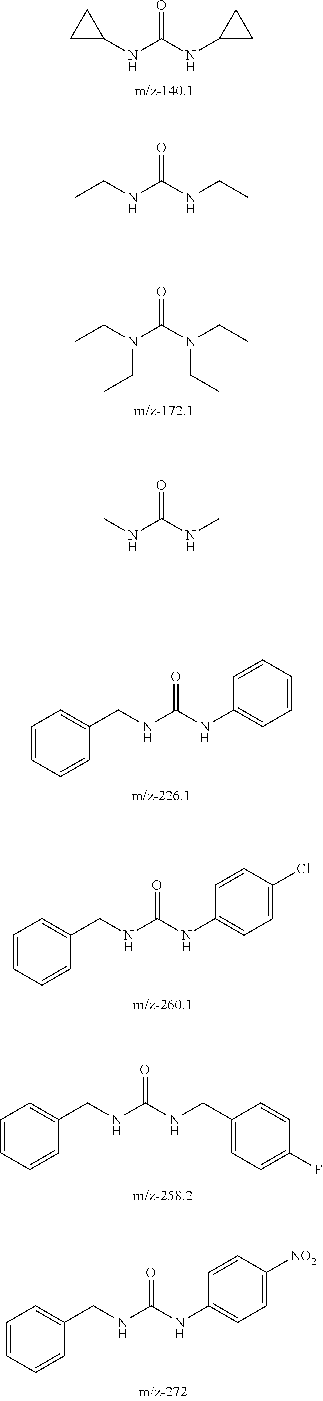 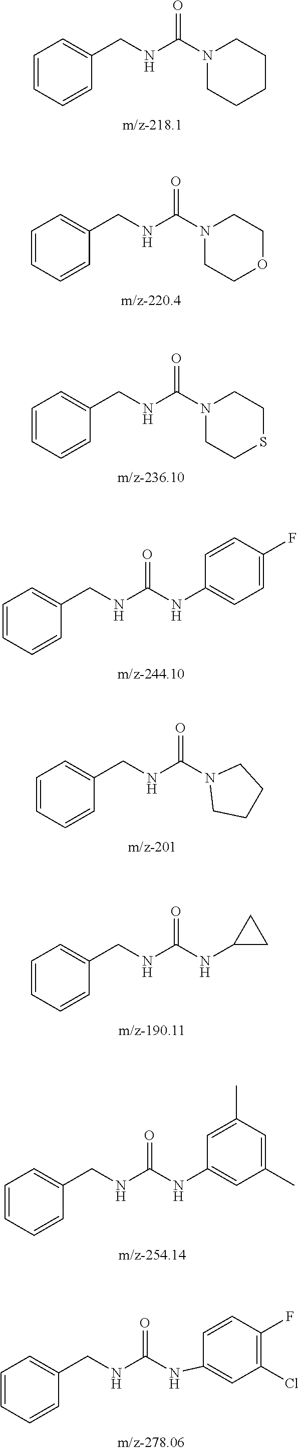 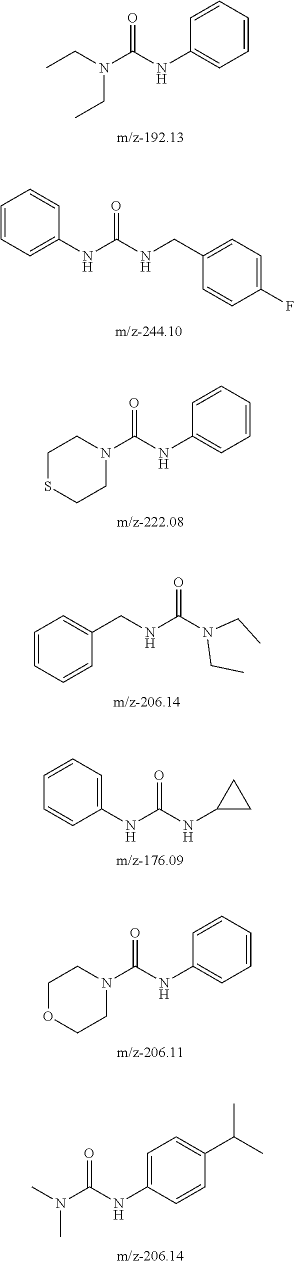 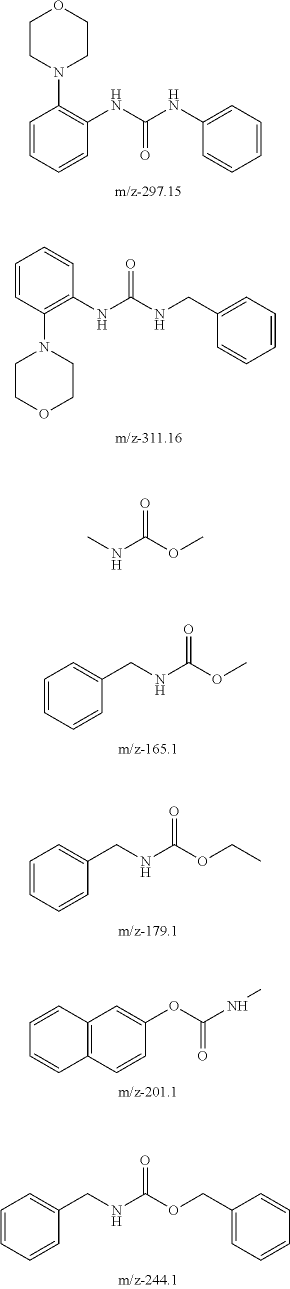 [chemical structure]
